(19) United States
(12) Patent Application Publication
(10) Pub. No.: US 2013/0115608 A1
(43) Pub. Date: May 9, 2013

(54) SCREENING METHODS FOR OCULAR IRRITATION AND TOXICITY

(71) Applicant: CeeTox, Inc., Kalamazoo, MI (US)

(72) Inventor: James M. McKim, Kalamazoo, MI (US)

(73) Assignee: CeeTox, Inc., Kalamazoo, MI (US)

(21) Appl. No.: 13/671,767

(22) Filed: Nov. 8, 2012

Related U.S. Application Data
(60) Provisional application No. 61/556,918, filed on Nov. 8, 2011.

(51) Int. Cl. C12Q 1/02

(52) U.S. Cl. CPC C12Q 1/025
USPC 435/6.12; 435/29

(57) ABSTRACT

Methods of determining a level of ocular irritation and/or toxicity for a chemical compound are described. Kits for use in methods of determining a level of ocular irritation and/or toxicity for a chemical compound are also described.

### STATEMENT REGARDING FEDERALLY SPONSORED RESEARCH OR DEVELOPMENT

Not Applicable.

### FIELD OF THE INVENTION

The present application for patent relates to in vitro methods for predicting in vivo toxicity of chemical compounds, including organ-specific toxicity of such chemical compounds, understanding the relative toxicity of said chemicals, and identifying mechanisms of toxicity.

### BACKGROUND OF THE INVENTION

The use of animals for safety testing is banned for chemicals intended for use in cosmetics. Personal care industries as well as regulators are forced to develop, adopt, and understand in vitro alternatives to animal models, such as the rabbit Draize ocular test and the rabbit Draize dermal test. Although there are several proposed in vitro methods for determining chemical toxicity in the eye, none of these encompass an integrative approach to data collection and analysis.

To date, evaluation of in vivo toxicity of a given candidate substance as a potential drug or cosmetic has involved the use of animal models. Ever since the introduction of the rabbit Draize test (Draize et al. 1944), personal care, cosmetic, chemical, and pharmaceutical companies have relied on this animal safety test to assess the ocular irritancy and toxicity of compounds. Due to recent legislative actions in the EU and growing concern in the United States, it is imperative that full replacements to the animal studies be developed, validated, and implemented. Underlying the animal tests is the assumption that the effects observed in animals are applicable and predictive of effects in humans. In general, when the dosage is based on per unit of body surface area, toxicology data from animals is applicable to humans. On the other hand, when the dosage is based on animal body weight, humans are typically more susceptible to toxicity than the test animals. Nevertheless, the vast majority of drugs are developed to be given on the basis of body weight.

Key issues in developing in vitro toxicity screens have been deciding on the type and nature of assays to be utilized and the test system to be employed. There are many biochemical and molecular assays that claim to assess toxicity in cells grown in culture. However, when only one or even two assays are used over a limited range of exposure concentrations, the probability of false negative and false positive data is high. Some of the most commonly used assays include, but are not limited to, leakage of intracellular markers as determined by lactate dehydrogenase (LDH), glutathione S-transferase (GST), and potassium, and the reduction of tetrazolium dyes such as MTT ((3-(4,5-Dimethylthiazol-2-yl)-2,5-diphenyltetrazolium bromide), XTT (sodium 2,3,-bis(2-methoxy-4-nitro-5-sulfophenyl)-5-[(phenylamino)-carbonyl]-2H-tetrazolium inner salt), Alamar Blue, and INT (2-(4-Iodophenyl)-3-(4-nitrophenyl)-5-phenyl-2H-tetrazolium chloride). All have been used as indicators of cell injury. Prior art in vitro toxicity screens typically only involve the use of one or two endpoints. The resulting data provides a yes/no or live/dead answer. This minimalist approach to the toxicity-screening problem has resulted in little progress towards developing a robust screening system capable of providing a useful toxicity profile that has meaning for predicting similar toxicity in animals. Therefore, there remains a need in the art for the development of new screening systems that provide more useful toxicity information, especially toxicity information that can be obtained rapidly and cost-effectively. A need exists for toxicity screening systems that do not require the use of animals but that provide reliable information on relative toxicity, mechanism of toxicity, and that effectively predict in vivo toxicity.

Personal hygiene products must be evaluated for potential adverse effects to consumers. In particular, the effects of new chemicals and products on the eye must be determined. A chemical's potential ocular corrosive or irritation properties must be evaluated. In the past, this has been done by using the rabbit Draize test (Draize et al., 1944). This is a qualitative test requiring the direct administration of the test substance into the eye of a rabbit followed by an evaluation of the cornea, iris, and conjunctivae. The resulting score from 0 to 110 (MMAS score) provides an indication of ocular toxicity. This test can result in false positives and on occasion even false negative data. Amendment VII to the Cosmetics Directive places a ban on the use of animals for the safety testing of chemicals used in cosmetics and bans the sale of final products that contain ingredients that may have been tested using animals. This means that tests like the Draize eye test in rabbits must be replaced with an alternative method that does not require the use of animals.

The development of a reconstructed human ocular model (such as but not limited to, those developed by SkinEthic Laboratories (Lyon, France) and MatTek Corporation (Ashland, Mass.)) provides an excellent test system for the development of new ocular toxicity screening platforms. This model is human based and histologically similar to the human eye cornea. This model in combination with the MTT assay is being proposed as an alternative method to the Draize test. Although correlative data is promising, the MTT assay is not based on a mechanism of pain (sensory input) or discomfort in the eye. Rather, it is based on a viability endpoint and is subject to compound interference, and the cell type in which it is used. Release of proinflammatory cytokines has also been proposed as an endpoint that can be used to predict ocular toxicity. Most work limited this to IL-1α detection, and the use of this endpoint has not provided the predictive resolution desired. There have been several alternative methods proposed (Abbott, 2005; Balls et al., 1999; Curren and Harbell, 1998; van Goethem et al., 2006 and Garle and Fry, 2003) for evaluating ocular toxicity. However, none has been validated and none take into account multiple endpoint analysis to identify true negatives and positives or to differentiate irritants from corrosives. To date, none of these methods has been validated as a replacement method for the Draize test, and thus there is a real and immediate need for more robust methods with endpoints that can be linked to mechanisms that underlie ocular inflammation, pain, and toxicity.

The cornea and conjunctivae are extensively innervated by neurons known to express the transient receptor potential vanilloid type 1 (TRPV1). TRPV1 has been reported to be a key component in the pain pathway (Murata and Masuka, 2006; Caterina et al., 1997). When stimulated, TRPV1 opens and allows divalent cations to enter the cell. An important ion affected is Ca²⁺. TRPV1 can be activated by chemicals that cause pain, and the influx of divalent ions is considered to be an initiating step in the pain pathway. A well known substrate for TRPV1 is capsaicin; if capsaicin is rubbed in the eyes, a burning sensation accompanied by inflammation is produced (Tominga et al., 1998; Trevisani et al., 2002). It has been proposed that the irritating properties of many commercially available soaps and surfactant containing personal care products may cause eye irritation via a similar mechanism. In fact, it has been shown that TRPV1 can be activated by many surfactants (Lilja et al. 2007; Lindegren et al., 2009). Activation of TRPV1 as a molecular indicator for pain has been proposed by Lindegren et al. (2009), in which SH-SYS5 cells stably transfected with TRPV1 is used as the model system (Lilja et al., 2007). In this test system, the cells expressing TRPV1 were exposed to chemicals, and Ca²⁺ influx was measured using FURA-2AM (Fura-2-acetoxymethyl ester) probe.

Based on the above, it is evident that there is a need in the art for new and improved in vitro methods of screening for ocular irritation and/or toxicity. It is to such novel toxicity screening systems that the presently disclosed and claimed inventive concept(s) is directed. The presently disclosed and claimed inventive concept(s) provides a significant improvement to existing technologies by combining multiple biochemical endpoints related to mechanisms of ocular toxicity including pain. Combining cell models also improves and expands the capabilities of this novel approach to data acquisition, analysis, and interpretation.

### BRIEF DESCRIPTION OF THE DRAWINGS

FIG. 1 graphically depicts the aspect of evaluating recovery of cell damage in the ocular irritation and toxicity method of the currently disclosed and claimed inventive concept(s).

### DETAILED DESCRIPTION OF EXEMPLARY EMBODIMENTS

The following detailed description and appendices describe and illustrate various exemplary embodiments. The description and drawings serve to enable one skilled in the art to make and use the presently disclosed and claimed inventive concept(s), and are not intended to limit the scope of the inventive concept(s) in any manner.

Relevant background information is available in the following US patents: U.S. Pat. No. 6,998,249 issued to McKim and Cockerell on Feb. 14, 2006; and U.S. Pat. No. 7,615,361, issued to McKim on Nov. 11, 2009. The entire contents of each of the above-referenced patents are expressly incorporated into this disclosure.

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of the presently disclosed and claimed inventive concept(s) have been described in terms of preferred embodiments, it will be apparent to those of skill in the art that variations may be applied to the compositions and/or methods and in the steps or in the sequence of steps of the method described herein without departing from the concept, spirit and scope of the presently disclosed and claimed inventive concept(s). All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

The term “about” is used to indicate that a value includes the inherent variation of error for the device, the method being employed to determine the value and/or the variation that exists among cell types/lines.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

The presently disclosed and claimed inventive concept(s) provides methods for determining if a chemical compound exhibits eye irritation and/or toxicity. Said methods combine biochemical and molecular endpoints and cell types to develop an effect profile that more accurately predicts chemical irritation and/or corrosion. The method utilizes an ocular cell type/line alone or in combination with another cell type/line, and a panel of key biochemical and molecular endpoints are measured in the cell type(s)/line(s) to predict ocular irritation and/or overall toxicity. For example but not by way of limitation, key pathways that control cell health, inflammatory response, pain, oxidative stress and cytoprotective responses are monitored in the cell type(s)/line(s); in addition, calcium influx is also monitored in the cell type(s)/line(s) as an indicator of pain/irritation/corrosion. Said methods may further utilize specific quantitative parameters determined from concentration response curves for the multiple endpoints measured. The methods may yet further include a determination of the time required to produce a half maximal effect (ET₅₀). ET₅₀ values provide a quantitative means of comparing responses across many test chemicals.

The cells utilized in accordance with the presently disclosed and claimed inventive concept(s) may be primary cells in culture or a cell line. The cells may be obtained from any mammalian source that is amenable to primary culture and/or adaptation into cell lines. In lieu of generating cell lines from animals, such cell lines may be obtained from, for example, American Type Culture Collection, (ATCC, Rockville, Md.), or any other Budapest treaty or other biological depository. The cells used in the assays may be from an animal source or may be recombinant cells tailored to express a particular characteristic of, for example, a particular disorder for which the drug development is being considered. In one embodiment, the cells are derived from tissue obtained from humans or other primates, rats, mice, rabbits, sheep, dogs and the like. Techniques employed in mammalian primary cell culture and cell line cultures are well known to those of skill in that art. Indeed, in the case of commercially available cell lines, such cell lines are generally sold accompanied by specific directions of growth, media and conditions that are preferred for that given cell line.

In one embodiment, the method utilizes an ocular cell line/type, such as but not limited to, freshly isolated primary mammalian ocular cells (isolated from human or other animal sources), a mammalian ocular cell line, or a human reconstructed ocular model (such as but not limited to, HCE (human corneal epithelium) produced by SkinEthic Laboratories (Lyon, France) or the EpiOcular™ model by MatTek Corporation (Ahsland, Mass.)). In one embodiment, the ocular cell line/type may be specific to the species in which the toxicity determination is required. For example, for determining human ocular irritation/toxicity, a primary culture of human ocular cells may be utilized; for canine ocular toxicity, a dog ocular cell line may be utilized. However, it is to be understood that the presently disclosed and claimed inventive concept(s) is not limited to the use of cells specific to the species in which the toxicity determination is required; for example, human cells may also predict animal effects, provided the animal possesses the same biochemical pathways and potential toxicity targets as humans.

The ocular cell type/line is cultured in the absence of the chemical compound as well as in the presence of a plurality of concentrations of said chemical compound (for example, but not by way of limitation, at exposure concentrations ranging from 0.01 to 2500 μM). At least two indicators of cell health/function are then measured at the plurality of concentrations of said chemical compound in the ocular cell type/line. Said indicators of cell health/function include, but are not limited to, cell viability, inflammatory response, oxidative stress, and cytoprotective pathways. The indicators of cell health/function may be measured at a single time point or multiple time points for each concentration of said chemical compound. In addition, the indicators of cell health/function may also be measured at a single time point or multiple time points during a recovery period (i.e., following exposure to the said chemical compound), as described in more detail herein below.

Cell viability may be measured by any methods described herein or otherwise known in the art, including but not limited to, lactate dehydrogenase (LDH) leakage, MTT reduction and combinations thereof.

Many eye irritants cause inflammatory responses. Therefore, the inflammatory response may be measured by any methods described herein or otherwise known in the art. In one non-limiting embodiment, the mRNA levels of a panel (i.e., at least two) of proinflammatory cytokines may be monitored by techniques described herein or known in the art, such as but not limited to, RT-PCR. By way of example but not limitation, the monitored proinflammatory genes may include genes encoding one or more of the following: IL-1α, TNF-α, TGF-3, IL-6, IL-8, iNOS, p53, and HSP proteins. In one non-limiting embodiment, total RNA may be isolated following multiple exposure periods (such as but not limited to, 1, 10, 20 30, 60 and 120 minute exposure periods), and quantitative RT-PCR may then be performed. Changes in the relative abundance of mRNA for each target gene are then measured and compared to vehicle controls (such as but not limited to, saline, DMSO or other appropriate agent).

Oxidative stress may be measured by any methods described herein or otherwise known in the art. As the production of reactive oxygen species has been linked to cell damage and toxicity, in one non-limiting example, the generation of reactive oxygen species may be measured using 7′-dichlorodihydrofluorescein diacetate (DCF-DA) and mitoSOX™ red mitochondrial superoxide indicator (Invitrogen Corporation, Carlsbad, Calif.). The redox state of the test system is then assessed by measuring glutathione (GSH/GSSG) ratios at each time point.

Cytoprotective pathways may be measured by any methods described herein or otherwise known in the art, such as but not limited to, Nuclear factor (erythroid-derived 2)-like 2 (Nrf2) expression/signaling. Cells under stress from reactive chemicals (electrophiles) activate Nrf2/Keap1 (Kelch-like ECH-associated protein 1) signaling receptors in the cytosol. Once activated, these nuclear transcription factors bind to the antioxidant response element (ARE) in the nucleus and induce the expression of mRNA coding for proteins that are cytoprotective. Genes controlled by Nrf2 include, but are not limited to, quinone oxidase, Aldoketo reductase, Aldehyde dehydrogenase, Thioredoxin, and GCLC (Glutamate-cysteine ligase catalytic subunit). The expression of one or more of these genes is measured to indicate Nrf2 expression/signaling, and thus activation of cytoprotective pathways.

In addition to the at least two indicators of cell health/function mentioned above, the potency of irritancy/discomfort/pain is measured for the chemical compound utilizing an indicator of calcium influx. Calcium influx may be measured by any methods described herein or otherwise known in the art, including but not limited to, TrpV1 activation. In a non-limiting example, activation of TRPV1 may be monitored using FURA-2AM.

If the ocular cell line/type described herein above expresses the indicator of calcium influx (for example, but not by way of limitation, TRPV1), then the indicator of calcium influx assay is conducted in combination with the assays for the at least two indicators of cell health/function (and using the same samples of ocular cell line/type cultured in the absence or presence of the plurality of concentrations of the chemical compound).

However, if the ocular cell line/type described herein above does not express the agent monitored in the calcium influx assay (i.e., the indicator of calcium influx), then a second cell type/line that expresses the monitoring agent is utilized. In this embodiment, said second cell type/line (that expresses the indicator of calcium influx) is cultured in the absence of the chemical compound as well as in the presence of a plurality of concentrations of said chemical compound (for example but not by way of limitation, at exposure concentrations ranging from 0.01 to 2500 μM). The indicator of calcium influx is then measured at the plurality of concentrations of said chemical compound in the second cell type/line. Non-limiting examples of second cell lines/types that express TRPV1 and that may be utilized in accordance with the presently disclosed and claimed inventive concept(s) include the bladder carcinoma cell line RT4 (Rigby et al., 1970) and the neuroblastoma cell line SH-SY5Y (Lilja et al., 2007; Lindegen et al., 2009).

Once the at least two indicators of cell health and the indicator of calcium influx are measured at the plurality of concentrations of the chemical compound, the response data obtained for each endpoint is plotted with response on the Y-axis and either exposure concentration or time on the X-axis. The resultant time and concentration response curves are then used to determine IC₅₀ and ET₅₀ values as well as IC₂₀ and IC₈₀, and ET₂₀ and ET₈₀ values (as described in more detail herein below). An algorithm designed to evaluate these data sets for potency, maximal effect, time of effect, number of endpoints responding, and the type of endpoint responding (as described in more detail herein below) is then used to analyze the data. The response profile obtained is compared to a database of chemicals that are known to be ocular toxicants. The chemicals in this database represent a broad spectrum of ocular toxicity. By comparing unknown chemical data profiles to known profiles, it is possible to generate a prediction of ocular toxicity. The unknown chemical can be labeled as an irritant or a corrosive, or labeled as non-toxic.

It is determined that the chemical compound exhibits ocular irritation and/or toxicity if at least one of the indicators of cell health/functions (and/or indicator of calcium influx) varies in cells cultured with one or more concentrations of the chemical compound when compared to cells cultured in the absence of the chemical compound.

A level of ocular specific irritation and/or toxicity of said chemical compound may also be determined for the chemical compound from the measurements described herein above by performing a concentration response analysis for at least one of the indicators of cell health/function (and/or indicator of calcium influx) from the measurements obtained at the plurality of concentrations of the chemical compound, and identifying from the concentration response analyses the highest concentration of said chemical compound at which no measurable toxic effect was observed for the at least one indicator of cell health/function (and/or indicator of calcium influx). Then an ocular specific toxic concentration can be selected as a concentration less than or equal to the highest concentration of said chemical compound at which no measurable toxic effect was observed for the at least one indicator of ocular cell health/function (and/or indicator of calcium influx). In addition, a concentration that produces a half maximal toxic effect (TC_{50 ocular}) may be determined for the at least one indicator of cell health/function.

In another embodiment of the presently disclosed and claimed inventive concept(s), the method includes measuring the effects of a compound both during exposure as well as during a recovery period following exposure. The measurement of the indicators of cell health/function during exposure periods do not allow the measurement of reversible changes caused by the compound, and thus do not provide a method of evaluating recovery of cell damage.

There is currently no method of evaluating recovery of cell damage in an ocular irritation assay. In the prior art ocular irritation assay, an eye model was exposed to the test compound for given exposure period(s) (such as but not limited to, 1, 4 and 24 hours), then rinsed with buffer. An MTT assay was then performed. As shown in Table I, based on the Draize score and ET₅₀ (the exposure time required to reduce MTT or cell viability by 50%), the test compound is classified as weak, moderate or severe. However, this approach is greatly flawed, because the measurement of MTT at the end of exposure is actually measuring cell death. Cell death is indicative of corrosion, not irritation; corrosion is an irreversible condition, while irritation is reversible.

TABLE 1

| Draize Score | Irritancy | ET₅₀ (Minutes) |
|---|---|---|
| 0-15 | Non-irritating, minimal | >265-26.5 |
| 15.1-25 | Mild | <26.5-11.7 |
| 25.1-50 | Moderate | <11.7-3.45 |
| 50.1-110 | Severe/Extreme | <3.45 |

The presently disclosed and claimed inventive concept(s) overcomes this defect of the prior art by measuring at least one indicator of cell health/function during a recovery period following exposure to the chemical compound. At the end of the exposure period(s) described herein above, the ocular cell line/type is washed (such as but not limited to, with buffer) to remove the chemical compound, and the cell line/type is allowed to recover for a period of time. At least one indicator of cell health/function is then measured at at least one concentration of said chemical compound in the ocular cell type/line (and may also be measured at the plurality of concentrations described herein above). Said indicator(s) of cell health/function may be measured at a single recovery time point or at multiple recovery time points for each concentration of said chemical compound.

In a non-limiting example, the at least one indicator of cell health/function is the mRNA level of at least one cytokine. At the end of the exposure period(s), the ocular cell line/type is washed with buffer to substantially remove all of the chemical compound, and allowed to recover for periods of 24 and 48 hours. mRNA levels of at least one cytokine are then measured.

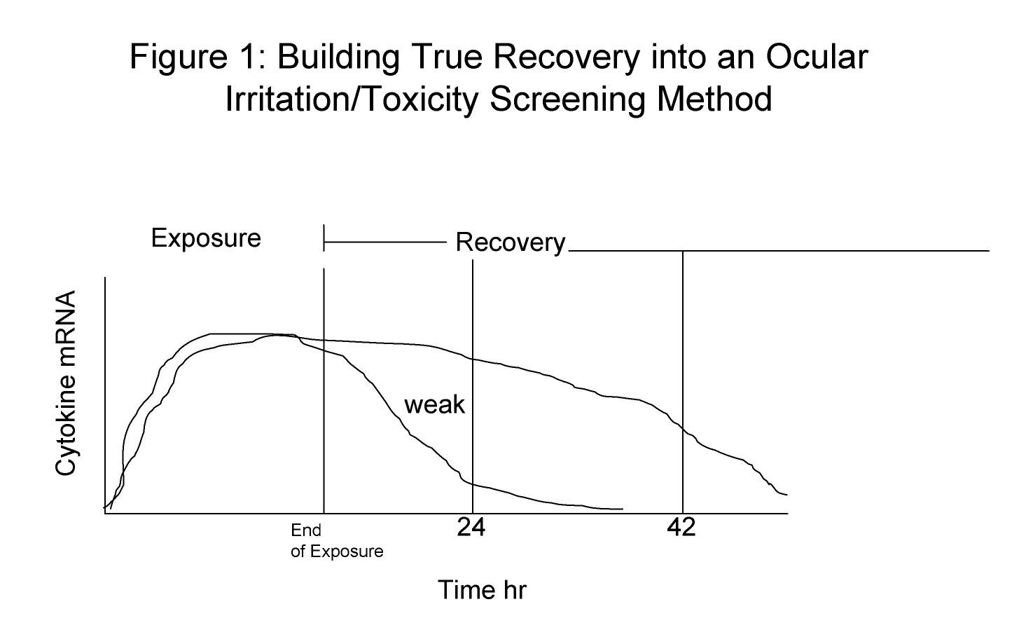

By measuring cytokine gene expression during exposure and recovery periods, cytokine mRNA induction and recovery can be measured; induction of cytokine gene expression will occur during exposure, while recovery of the cytokine gene expression induction may be seen during the recovery period (see FIG. 1). Measurements of cytokine gene expression (or other indicator(s) of cell health/function) during both exposure and recovery periods render it possible to delineate between a true irritation and a cell killing effect.

By further combining the exposure and recovery data with the measurements of potency or irritancy/discomfort/pain, it is possible to accurately differentiate the potency of an irritant based on mechanism and provide a much more comprehensive picture of ocular irritation.

Turning now to the particulars of certain embodiments of the presently disclosed and claimed inventive concept(s), the cells utilized may be seeded in multiwell (e.g., 96-well) plates and allowed to reach log phase growth. Once the cell cultures are thus established, various concentrations of the compound being tested are added to the media, and the cells are allowed to grow exposed to the various concentrations for a period of time, such as 2 hours. While the 2 hour exposure period is described, it should be noted that this is merely an exemplary time of exposure, and testing the specific compounds for longer or shorter periods of time is contemplated to be within the scope of the inventive concept(s). As such it is contemplated that the cells may be exposed to the test compound for 1, 10, 20, 30, 60, 120 or more minutes. Increased culture times may sometimes reveal additional cytotoxicity information, at the cost of slowing down the screening process.

Furthermore, the cells may be exposed to the test compound at any given phase in the growth cycle. For example, in some embodiments, it may be desirable to contact the cells with the compound at the same time as a new cell culture is initiated. Alternatively, it may be desirable to add the compound when the cells have reached confluent growth or arc in log growth phase. Determining the particular growth phase cells are in is achieved through methods well known to those of skill in the art.

The varying concentrations of the given test compound are selected with the goal of including some concentrations at which no toxic effect is observed and also at least two or more higher concentrations at which a toxic effect is observed. A further consideration is to run the assays at concentrations of a compound that can be achieved in vivo. For example, assaying several concentrations within the range from 0 micromolar to about 2500 micromolar is commonly useful to achieve these goals. The estimated therapeutically effective concentration of a compound provides initial guidance as to upper ranges of concentrations to test. Additionally, as explained in greater detail below, CATS analysis may further include assaying a range of concentrations that includes at least two concentrations at which cytotoxicity is observable in an assay. It has been found that assaying a range of concentrations as high as 2500 micromolar often satisfies this criterion.

In an exemplary set of assays, the test compound concentration range under which the CATS is conducted (and which also apply to any of the methods described and claimed herein) comprises dosing solutions which yield final growth media concentration of 0.01 micromolar, 0.1 micromolar, 1.0 micromolar, 5.0 micromolar, 10.0 micromolar, 20.0 micromolar, 50.0 micromolar, 100 micromolar, 300 micromolar, 500 micromolar, 1000 micromolar, 1500 micromolar, 2000 micromolar and 2500 micromolar of the compound in culture media. As mentioned, these are exemplary ranges, and it is envisioned that any given assay will be run in at least two different concentrations, and the concentration dosing may comprise, for example, 4, 5, 6, 7, 8, 9, 10, 11, 12, 13, 14, 15 or more concentrations of the compound being tested. Such concentrations may yield, for example, a media concentration of 0.05 micromolar, 0.1 micromolar, 0.5 micromolar, 1.0 micromolar, 2.0 micromolar, 3.0 micromolar, 4.0 micromolar, 5.0 micromolar, 10.0 micromolar, 15.0 micromolar, 20.0 micromolar, 25.0 micromolar, 30.0 micromolar, 35.0 micromolar, 40.0 micromolar, 45.0 micromolar, 50.0 micromolar, 55.0 micromolar, 60.0 micromolar, 65.0 micromolar, 70.0 micromolar, 75.0 micromolar, 80.0 micromolar, 85.0 micromolar, 90.0 micromolar, 95.0 micromolar, 100.0 micromolar, 110.0 micromolar, 120.0 micromolar, 130.0 micromolar, 140.0 micromolar, 150.0 micromolar, 160.0 micromolar, 170.0 micromolar, 180.0 micromolar, 190.0 micromolar, 200.0 micromolar, 300 micromolar, 400 micromolar, 500 micromolar, 600 micromolar, 700 micromolar, 800 micromolar, 900 micromolar, 1000 micromolar, 1100 micromolar, 1200 micromolar, 1300 micromolar, 1400 micromolar, 1500 micromolar, 1600 micromolar, 1700 micromolar, 1800 micromolar, 1900 micromolar, 2000 micromolar, 2100 micromolar, 2200 micromolar, 2300 micromolar, 2400 micromolar, and 2500 micromolar in culture media. It will be apparent that a cost-benefit balancing exists in which the testing of more concentrations over the desired range provides additional information, but at additional cost, due to the increased number of cell cultures, assay reagents, and time required. In one embodiment, ten different concentrations over the range of 0 micromolar to 2500 micromolar are screened.

Typically, the various assays described in the present specification may employ cells seeded in 96 well plates or 384 cell plates. The cells are then exposed to the test compounds over a concentration range, for example, 0-2500 micromolar. The cells are incubated in these concentrations for a given period of, for example, 1 minute, 10 minutes, 20 minutes, 30 minutes, 60 minutes, and/or 120 minutes. Subsequent to the incubation, the assays of the cluster are performed for each test compound. In one embodiment, all the assays are performed at the same time such that a complete set of data are generated under similar conditions of culture, time and handling. However, it may be that the assays are performed in batches within a few days of each other.

The compounds to be tested may include fragments or parts of naturally-occurring compounds or may be derived from previously known compounds through a rational drug design scheme. It is proposed that compounds isolated from natural sources, such as animals, bacteria, fungi, plant sources, including leaves and bark, and marine samples may be assayed as candidates for the presence of potentially useful pharmaceutical compounds. Alternatively, pharmaceutical compounds to be screened for toxicity could also be synthesized (i.e., man-made compounds).

The types of compounds being monitored may be antiviral compounds, antibiotics, anti-inflammatory compounds, antidepressants, analgesics, antihistamines, diuretic, antihypertensive compounds, antiarrythmia drugs, chemotherapeutic compounds for the treatment of cancer, antimicrobial compounds, among others.

Regardless of the source or type of the compound to be tested for cytotoxicity, it may be necessary to monitor the biological activity of the compounds to provide an indication of the therapeutic efficacy of a particular compound or group of compounds. Of course, such assays will depend on the particular therapeutic indication being tested. Exemplary indications include efficacy against Alzheimer's disease, cancer, diabetes, depression, immunodeficiency, autoimmune disease, inflammatory disease and the like.

Cluster Analyses Assays: The use of multiple assays to develop a toxicity profile for new drugs proves to be a very powerful tool for accurately assessing the effects of a compound in a living system.

Selective assays used in the clusters of the presently disclosed and claimed inventive concept(s) provide key information pertaining to the ocular irritation and/or toxicity profile of a given compound. The assays may be performed such that information regarding the various parameters is obtained at the same time during the drug development phase of drug discovery as opposed to performing the assays at different times during the drug development scheme. In one embodiment, the assays are performed in a batch all at the same time. In other aspects, it may be useful to perform the assays on cell cultures all generated at the same time from an initial cell line.

Modules may be designed in which a cluster of assays address a specific concern. Thus, in order to monitor the effect of a specific compound on the general health of a cell, monitoring membrane integrity, mitogenesis, mitochondrial function and energy balance will be particularly useful. The specific assay employed for any of these endpoints is not considered to be limiting. Thus, any assay that provides an indication of membrane integrity may be combined with any assay that is predictive of mitogenesis (cell replication) along with any assay that is an indicator of mitochondrial function and energy balance.

In addition to a module for determining the general cell health, other modules of interest would include those that are directed to determining for example, oxidative stress, cell cycle parameters, acute inflammatory response, apoptosis, endocrine responses and interaction with cell membrane transporters such as Pgp.

In a module that determines oxidative stress, production of reactive oxygen species (ROS), reactive nitrogen species (RNS), or lipid peroxidation may be monitored. Exemplary assays to be employed in the cluster may involve monitoring endpoints that include but are not limited to glutathione/glutathione disulfide (GSH/GSSG), dichlorofluoroscindiacetate (DCFDA), lipid peroxidation, 8-isoprostane, 8-oxy guanine (8-oxy G) DNA adducts, thiobarbituric acid (TBARS), and malondialdehyde (MDA).

Modules designed to monitor cell cycle may include determining the effect on the presence or level of any given cell cycle indicator including but not limited to p53, p21, TGFβ, CDK1, PCNA, telomerase, nitric oxide, and inducible nitric oxide synthase (iNOS). Again any particular assay may be employed to determine the level or amount of any given cell cycle indicator.

Modules to monitor apoptosis may include any assays described herein or otherwise known in the art. One example of such an assay is a caspase-3 assay; however, the presently disclosed and claimed inventive concept(s) is to be understood to not be limited to the use of such assay, and any apoptosis assay may be substituted therefor in accordance with the presently disclosed and claimed inventive concept(s).

As stated above, the specific assay to monitor any of the given parameters is not considered crucial so long as that assay is considered by those of skill in the art to provide an appropriate indication of the particular biochemical or molecular biological endpoint to be determined, such as information about mitochondrial function, energy balance, membrane integrity, cell replication, and the like. Exemplary assays that may be used in the context of the presently disclosed and claimed inventive concept(s) can be found in the inventor's U.S. Pat. Nos. 6,998,249 and 7,615,361, previously incorporated herein by reference. However, the disclosures of said patents are not intended to be an exhaustive treatise on the description of these assays, but rather to be a guide as to the type of assays that are available to those of skill in the art. A person having ordinary skill in the art will easily recognize assays that are known in the art that may be utilized in accordance with the presently disclosed and claimed inventive concept(s).

Predicting In vivo Toxicity of a Compound from In vitro Analyses: Once all data for a given cluster of assays are received, the data are analyzed to obtain a detailed profile of the compound's toxicity. For example, most conveniently, the data are collated over a dose response range on a single graph. In such an embodiment, the measurement evaluated for each parameter (i.e., each indicator of cell health) at any given concentration is plotted as a percentage of a control measurement obtained in the absence of the compound. However, it should be noted that the data need not be plotted on a single graph, so long as all the parameters are analyzed collectively to yield detailed information of the effects of the concentration of the compound on the different parameters to yield an overall toxicity profile. As set forth below, this overall toxicity profile will facilitate a determination of a plasma concentration, Cₜₒₓ that is predicted to be toxic in vivo. Cₜₒₓ represents an estimate of the sustained plasma concentration in vivo that would result in toxicity, such as hepatotoxicity or hematopoietic toxicity.

A fundamental premise in the field of toxicology is that all compounds are poisons, and that it is the dose of the compound that determines a beneficial/therapeutic effect versus a toxic effect. Dose is affected by time of exposure, dosing regimens, pharmacokinetic parameters such as absorption, metabolism and elimination, by difference between species being treated, and by route of administration. All these factors influence the plasma concentration of a drug and its duration of exposure. Thus, in principle, in vitro screens need only account for metabolism and time of exposure. In theory, an increased exposure time should shift the dose response curve to the left (e.g., TC₅₀ is lower or the compound appears more toxic over longer exposure times). These factors all have been considered in the selection of Cₜₒₓ in the CATS assay.

In the specific embodiments, the results of the analyses are depicted on a single graph on which the values are presented relative to control. The term “relative to control” means that the measurements in the presence of a given concentration of the compound are compared to a similar assay performed in the absence of the compound. The measurement in the absence of the compound is presented as the 100% measurement. The effect of the compound is thus determined as a raw figure which is then adjusted relative to that measurement that is determined in the absence of the compound.

The presently disclosed and claimed inventive concept(s) also includes a method of predicting ocular toxicity. In said method, a database of drugs with varying ocular toxic potencies is utilized to assess data for a chemical compound of unknown toxicity (wherein the data is obtained as described herein above). Maximum therapeutic plasma concentrations (Cₘₐₓ) are compared to ocular specific markers and the concentration that produces half maximal toxicity (EC₅₀ or TC₅₀). Ocular specific toxicity is then predicted. Note that as Cₘₐₓ or plasma concentration exceeds the EC₅₀ of the indicator of ocular specific cell health, the probability of toxicity increases.

The database allows for a three dimensional scatter plot of many known drugs with known ocular toxicity to be plotted with their plasma concentration data. The quadrants formed provide a good indication of ocular toxicity in humans.

Specific examples of assays and analyses that may be utilized in accordance with the presently disclosed and claimed inventive concept(s) may be found in U.S. Pat. Nos. 6,998,249 and 7,615,361, and previously incorporated herein by reference, as well as in U.S. Ser. No. 61/355,633, filed Jun. 17, 2010 (the entire contents of which are expressly incorporated herein by reference).

In certain aspects of the presently disclosed and claimed inventive concept(s), all the necessary components for conducting one or more of the assays described herein above may be packaged into a kit. Specifically, the presently disclosed and claimed inventive concept(s) provides a kit for use in an assay for ocular specific irritation and/or toxicity. The kits comprise packaged sets of reagents for conducting one or more cell health/function assay(s), as described in detail herein above. For example but not by way of limitation, said kits may comprise packaged sets of reagents for conducting at least one cell health/function assay selected from the group consisting of a cell viability assay, an inflammatory response assay, a calcium influx assay, an oxidative stress assay and a cytoprotective pathway assay. In addition to the reagents, the kit may also include instructions packaged with the reagents for performing one or more variations of the assay(s) of the presently disclosed and claimed inventive concept(s) using the reagents. The instructions may be fixed in any tangible medium, such as printed paper, or a computer-readable magnetic or optical medium, or instructions to reference a remote computer data source such as a worldwide web page accessible via the internet.

While the above embodiments contemplate kits in which there is at least one specific cell health/function assay, it is contemplated that the kits and the methods may involve conducting more than one specific cell health/function assay. As such, it is contemplated that the kits also may comprise the reagents for conducting a second assay from each of the classes.

Thus, in accordance with the presently disclosed and claimed inventive concept(s), there have been provided methods of determining a level of ocular irritation and/or toxicity for a chemical compound that fully satisfy the objectives and advantages set forth hereinabove. Although the inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the inventive concept(s).

What is claimed is:

1. A method for determining if a chemical compound exhibits ocular irritation and/or toxicity, the method comprising the steps of:
culturing an ocular cell line/type in the absence of the chemical compound and in the presence of a plurality of concentrations of said chemical compound;
measuring at least two indicators of cell health/function in the ocular cell line/type in the absence of the chemical compound and in the presence of the plurality of concentrations of said chemical;
measuring a potency of irritancy/discomfort/pain in the ocular cell line/type in the absence of the chemical compound and in the presence of the plurality of concentrations of said chemical compound, wherein the potency is measured utilizing an indicator of calcium influx and the ocular cell line/type expresses the indicator of calcium influx; and
determining that the chemical compound exhibits ocular irritation and/or toxicity if at least one of the indicators of cell health/function and calcium influx varies in cells cultured with one or more concentrations of the chemical compound when compared to cells cultured in the absence of the chemical compound.

2. The method of claim 1, wherein the ocular cell line/type is selected from the group consisting of freshly isolated primary mammalian ocular cells, a mammalian ocular cell line, a human reconstructed ocular model, and combinations thereof.

3. The method of claim 1, wherein the ocular cell line/type is specific to the species in which the toxicity determination is required.

4. The method of claim 1, wherein the at least two indicators of cell health/function are selected from the group consisting of cell viability, inflammatory response, oxidative stress, cytoprotective pathways and combinations thereof.

5. The method of claim 4, wherein at least one of:
(a) an indicator of cell health/function is an indicator of cell viability selected from the group consisting of LDH leakage, MTT reduction and combinations thereof;
(b) an indicator of cell health/function is an indicator of inflammatory response selected from the group consisting of mRNA levels of at least two proinflammatory cytokines selected from the group consisting of IL-1α, TNF-α, TGF-β, IL-6, IL-8, iNOS, p53, and HSP proteins;
(c) an indicator of cell health/function is an indicator of oxidative stress selected from the group consisting of 7′-dichlorodihydrofluorescein diacetate (DCF-DA) and/or red mitochondrial superoxide indicator in combination with glutathione (GSH/GSSG) ratios;
(d) an indicator of cell health/function is an indicator of cytoprotective pathways further defined as analysis of Nrf2 expression/signaling; and
(e) an indicator of calcium influx comprises TRPV1 activation.

6. The method of claim 1, further comprising the step of measuring the at least two indicators of cell health/function and/or the indicator of calcium influx at multiple time points for each concentration of the chemical compound.

7. The method of claim 1, further comprising the step of measuring at least one of the indicators of cell health/function/calcium influx at at least one time point during a recovery period following exposure to the chemical compound and washing of the ocular cell line/type to substantially remove the chemical compound.

8. The method of claim 1, further comprising the steps of:
performing a concentration response analysis for at least one of the indicators of cell health/function and calcium influx from the measurements obtained at the plurality of concentrations of the chemical compound; and
identifying from the concentration response analysis the highest concentration of said chemical compound at which no measurable toxic effect was observed for the at least one indicator of cell health/function/calcium influx.

9. The method of claim 8, further comprising the steps of:
determining a concentration that produces a half maximal toxic effect (TC₅₀) for at least one indicator of cell health/function; and
determining a concentration that produces a half maximal toxic effect (TC₅₀) for the indicator of calcium influx.

10. The method of claim 1, wherein the plurality of concentrations of the chemical compound are in a range of from 0.01 micromolar to 2500 micromolar.

11. The method of claim 1, further comprising the step of measuring the biological activity of the chemical compound at the plurality of concentrations of said chemical compound in the ocular cell type/line to provide an indication of therapeutic efficacy of the chemical compound.

12. The method of claim 1, further comprising plotting the measurements for each said cell health/function/calcium influx indicator on a graph as a function of concentration for each said cell health/function/calcium influx indicators of the chemical compound to create a response profile for each indicator.

13. The method of claim 12, further comprising the step of calculating a control measurement, and wherein the measurements of each of said cell health/function/calcium influx indicators are expressed relative to the control measurement as a function of concentration of the chemical compound.

14. A method for determining if a chemical compound exhibits ocular irritation and/or toxicity, the method comprising the steps of:
culturing an ocular cell line/type in the absence of the chemical compound and in the presence of a plurality of concentrations of said chemical compound;
culturing a second cell line/type that expresses an indicator of calcium influx in the absence of the chemical compound and in the presence of a plurality of concentrations of said chemical compound;
measuring at least two indicators of cell health/function in the ocular cell line/type in the absence of the chemical compound and in the presence of the plurality of concentrations of said chemical compound;
measuring a potency of irritancy/discomfort/pain in the second cell line/type in the absence of the chemical compound and in the presence of the plurality of concentrations of said chemical compound utilizing an indicator of calcium influx; and
determining that the chemical compound exhibits ocular irritation and/or toxicity if at least one of the indicators of cell health/function and calcium influx varies in cells cultured with one or more concentrations of the chemical compound when compared to cells cultured in the absence of the chemical compound.

15. A kit for use in an ocular specific irritation and/or toxicity assay, the kit comprising:
at least one reagent for measuring a first indicator of cell health/function in an ocular cell type/line;
at least one reagent for measuring a second indicator of cell health/function in an ocular cell type/line; and
at least one reagent for measuring an indicator of calcium influx in an cell type/line that expresses the indicator of calcium influx.

16. The kit of claim 15, wherein the two indicators of cell health/function ire selected from the group consisting of a cell viability assay, an inflammatory response assay, an oxidative stress assay, a cytoprotective pathway assay, and combinations thereof.

17. The kit of claim 15, further comprising instructions for performing one or more variations of the assay(s).
